(19) United States
(12) Patent Application Publication
(10) Pub. No.: US 2023/0148370 A1
(43) Pub. Date: May 11, 2023

(54) ERA PATIENT MONITOR

(71) Applicants: Eras Lucknow Medical College & Hospital, Lucknow, (IN); American University of Barbados, Bridgetown (BB)

(72) Inventors: Mohsin Ali Khan, Lucknow (IN); Zaw Ali Khan, Lucknow (IN); Sarina Zehra, Lucknow (IN); Kinza Zehra, Lucknow (IN)

(21) Appl. No.: 18/150,321

(22) Filed: Jan. 5, 2023

(51) Int. Cl. A61B 5/00; G16H 70/60; G16H 80/00

(52) U.S. Cl. CPC A61B 5/002; A61B 5/6826; A61B 5/746; A61B 5/742; A61B 5/0024; G16H 70/60; G16H 80/00

(57) ABSTRACT

The present disclosure relates to an era patient monitor. In particular, the preset disclosure provides a portable patient health monitor system, wherein the system comprises: plurality of sensor input lids; a controlling unit; a communication device; and a monitor. The proposed monitor measures the real time data, and provide exact measurement of various health parameters namely SpO2, Pulse rate, Heart Rate, ECG, temperature, invasive blood pressure (IBP), and Respiration rate of patient. The proposed monitor has a bi-directional integration facility using Wi-Fi/LAN. The monitor also has an alert feature, which will indicate any kind of abnormality, if found. The proposed monitor also has video and audio calling feature, along with various other important feature, thus making it more efficient and effective solution as a portable patient health monitoring system.

### TECHNICAL FIELD

The present disclosure relates an era patient monitor. More particularly, the present disclosure relates to a portable patient health monitoring system that measures the exact SpO2, Pulse rate, Heart Rate, ECG, Respiration rate of patient, and some other health parameters of patient.

### BACKGROUND

Clinical monitoring systems have been developed to help healthcare providers monitor the vital signs of multiple patients in different locations at the same time. These systems allow for the collection of data on important bodily functions such as temperature, pulse, and heart rate, and enable healthcare providers to identify changes in a patient's condition and assess their overall condition over time. This is particularly useful for patients who require moderate to high levels of care and for hospitals and other clinical facilities that care for multiple patients in separate rooms, as it can be challenging for a limited number of clinicians to monitor all of these patients consistently.

To address this issue, some clinical testing frameworks have been created to enable the basic signs information gathered from patients to be passed on to a focal point, allowing one or two clinicians to screen numerous patients in various areas at the same time.

In any event, a big number of previous frameworks did not allow the checked patients to wander around the clinic. Although a few of “portable” observation frameworks have been attempted, such frameworks are difficult to use and prone to failure, resulting in the transmission of a patient's vital physiological functions information.

In prior arts U.S. Pat. No. 6,544,173B2 and USD725784S1, a wireless medical telemetry system is proposed that consists of at least one wireless patient monitor capable of collecting patient vital signs data and at least one central station capable of communicating with the at least one patient monitor via a wireless transceiver and receiving patient vital signs data from the at least one patient monitor.

In another prior art US-20080194918-A1, a system and device are suggested for monitoring at least one physiological parameter of a patient and selecting providing entertainment material and at least one physiological parameter on a common display.

However the above mentioned prior arts have certain limitations which can be alleviated by developing an efficient portable health monitoring system.

In the view of the foregoing discussion, it is clearly portrayed that there is a need of a portable patient health monitoring system that measures the exact SpO2, Pulse rate, Heart Rate, ECG, Respiration rate of patient, and some other health parameters of patient.

### BRIEF SUMMARY

The present disclosure relates to an era patient monitor which is a portable patient health monitoring system that measures the exact SpO2, Pulse rate, Heart Rate, ECG, Respiration rate of patient, and some other health parameters of patient. The proposed era patient monitor has the capability of video calling and real-time data transfer of patients to doctors and attendants via RMD/Dig doctor software. The proposed monitor supports bidirectional integration through Wi-Fi/LAN. If the monitor detects any irregularities in the measuring parameters, it will display an abnormality indicator and an alarm. The various feature of the proposed monitor includes Real-Time Monitoring Data, Video and Audio Calling, Facilities for Bi-Directional Integration, Connectivity (wired and wireless), A Real-Time View of an ECG Graph, Abnormality Alarms and Indicators, feature to review the most recent pathology and biochemistry results, and Monitoring ABG report.

The present disclosure seeks to provide a portable patient health monitoring system. The system comprises: a plurality of sensor input lids engaged to the fingertips of a person/patient for detecting a set of health parameters; a controlling unit for generating an alert signal upon comparing the detected set of health parameters to the threshold values stored in a database; a communication device having a bi-directional integration facility for transferring the alert signal to a respective alarm indicator from a plurality of alarm indicators for indicating an imbalance of respective health parameters from the set of health parameters; and a monitor for displaying the detected set of health parameters and indicating the imbalanced health parameters from the set of health parameters, wherein the graphical user interface allows checking the last pathology & Biochemistry, ABG report.

In an embodiment, the system further comprises a camera for supporting video calling and real-time data transfer of patients to the doctor, and attendants using an RMD/Dig doctor user interface

In an embodiment, the set of health parameters is selected from a group of exact oxygen saturation (SpO2), Pulse rate, Heart Rate, electrocardiography (ECG), temperature, Invasive Blood Pressure (IBP) and Respiration rate of the patient.

In an embodiment, the system further comprises a graphical user interface for displaying the detected set of health parameters and indicates the imbalanced health parameters from the set of health parameters, wherein the graphical user interface allows checking the last pathology & Biochemistry, ABG report remotely.

In an embodiment, the system further comprises an internet medium preferably a Wi-Fi to allow clinicians to make informed decisions, identify, and alert.

In an embodiment, the detected set of health parameters is converted from analog data into digital data using serial communication.

In an embodiment, the system further comprises an audio calling facility through the graphical user interface or through the communication module.

In an embodiment, the system is optionally paired with a user computing device to support real-time video calling with the doctor.

In an embodiment, the system further comprises a cloud server platform wirelessly connected to the controlling unit for storing the database of the threshold values.

An objective of the present disclosure is to provide an era patient monitor which is a portable patient health monitoring system.

Another object of the present disclosure is to provide a system that can measure the exact values of health parameters of patient such as SpO2, Pulse rate, Heart Rate, ECG, temperature, invasive blood pressure (IBP), and Respiration rate of patient.

Yet, another object of the present disclosure is to provide the accurate and portable patient health monitoring system

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a block diagram of a portable patient health monitoring system in accordance with an embodiment of the present disclosure; and

FIGS. 2A and 2B illustrate a flow chart and block diagram of an era patient monitor in accordance with an embodiment of the present disclosure.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

In this respect, before explaining at least one embodiment of the invention in detail, it is to be understood that the invention is not limited in its application to the details of construction and to the arrangements of the components set forth in the following description.

The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure.

It is intended that the following detailed description be considered as exemplary only, with the true scope and spirit being indicated by the following claims. Additional illustrative embodiments are listed below.

In today's multifaceted approach to healthcare, the demands on clinicians are massive to provide consistency and reduce complexity; the current invention relates to a Portable Patient Monitor equipped with Wi-Fi (Internet) capability to assist clinicians in making informed decisions, identifying and alerting patients.

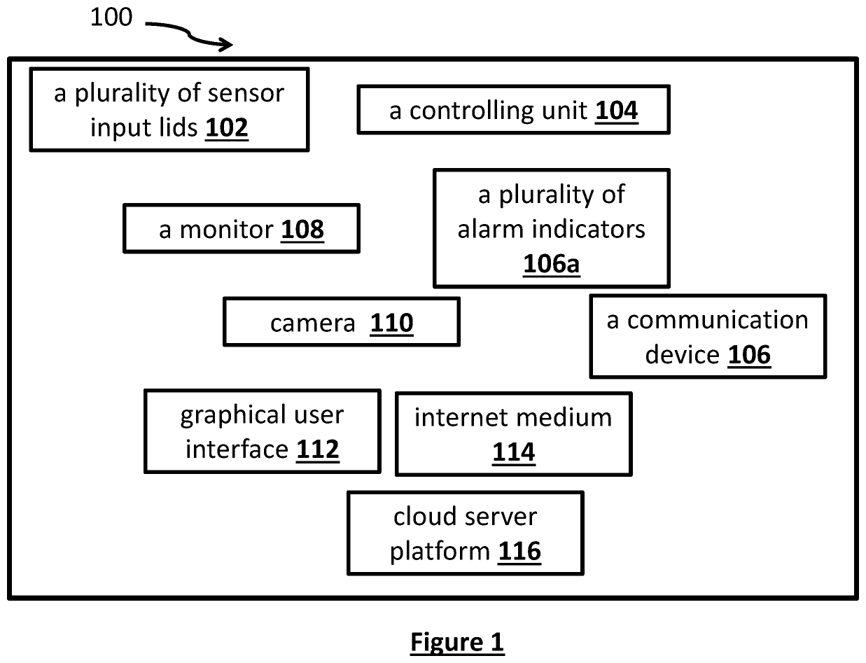

FIG. 1 illustrates a block diagram of a portable patient health monitoring system in accordance with an embodiment of the present disclosure. The system 100 includes a plurality of sensor input lids (102) engaged to the fingertips of a person/patient for detecting a set of health parameters.

In an embodiment, a controlling unit (104) is utilized for generating an alert signal upon comparing the detected set of health parameters to the threshold values stored in a database.

In an embodiment, a communication device (106) have a bi-directional integration facility for transferring the alert signal to a respective alarm indicator from a plurality of alarm indicators (106a) for indicating an imbalance of respective health parameters from the set of health parameters.

In an embodiment, a monitor (108) is utilized for displaying the detected set of health parameters and indicating the imbalanced health parameters from the set of health parameters, wherein the graphical user interface allows checking the last pathology & Biochemistry, ABG report.

In an embodiment, a camera (110) is employed for supporting video calling and real-time data transfer of patients to the doctor, and attendants using an RMD/Dig doctor user interface.

In an embodiment, the set of health parameters is selected from a group of exact oxygen saturation (SpO2), Pulse rate, Heart Rate, electrocardiography (ECG), temperature, Invasive Blood Pressure (IBP) and Respiration rate of the patient.

In an embodiment, a graphical user interface (112) is employed for displaying the detected set of health parameters and indicates the imbalanced health parameters from the set of health parameters, wherein the graphical user interface allows checking the last pathology & Biochemistry, ABG report remotely.

In an embodiment, an internet medium (114) is employed preferably a Wi-Fi to allow clinicians to make informed decisions, identify, and alert.

In an embodiment, the detected set of health parameters is converted from analog data into digital data using serial communication.

In an embodiment, an audio calling facility is utilized through the graphical user interface or through the communication module.

In an embodiment, the system (100) is optionally paired with a user computing device to support real-time video calling with the doctor.

In an embodiment, a cloud server platform (116) is wirelessly connected to the controlling unit for storing the database of the threshold values.

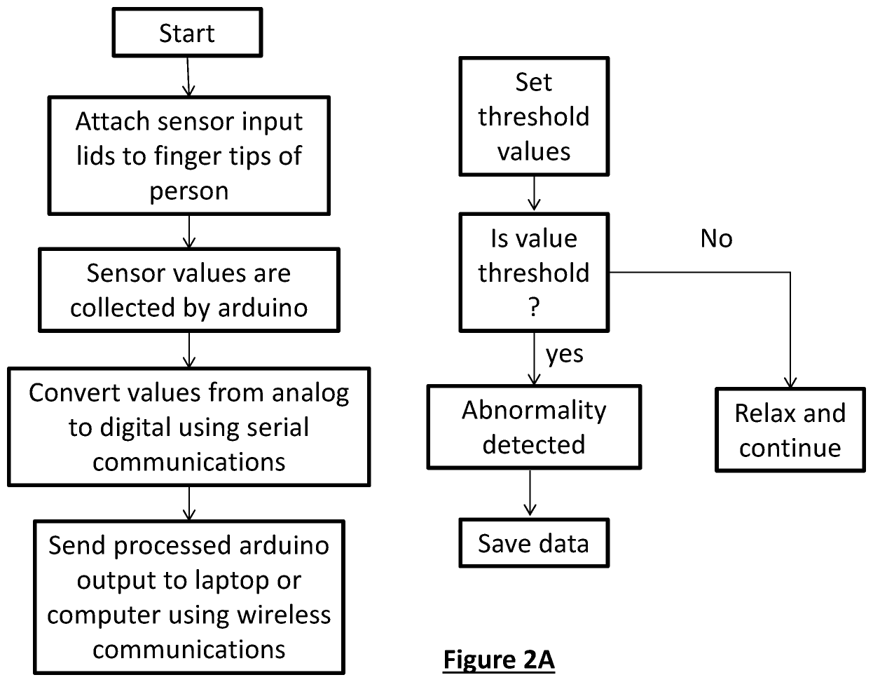

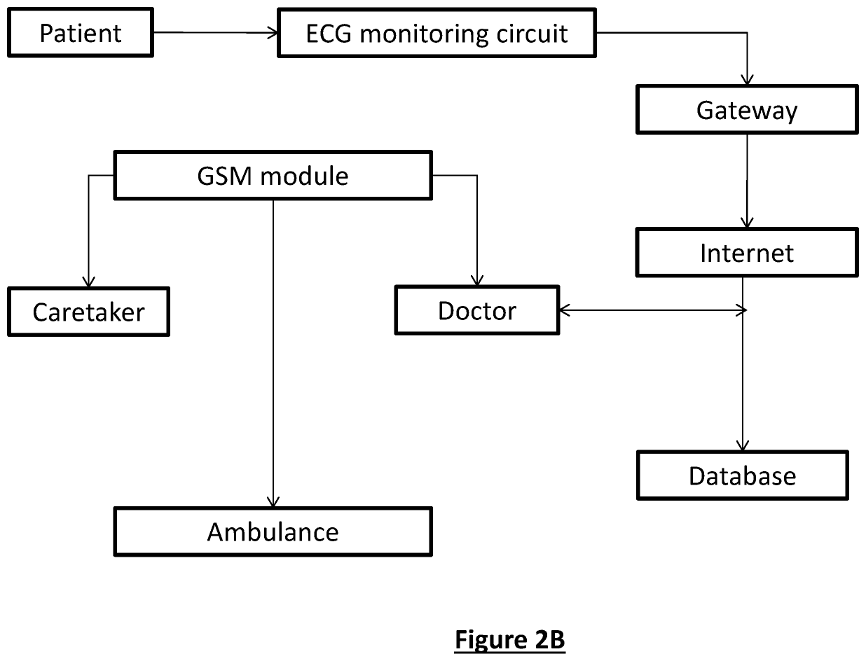

FIGS. 2A and 2B illustrates a flow chart and block diagram of an era patient monitor in accordance with an embodiment of the present disclosure.

In the proposed monitor, the facility of video calling and real time data transfer of patient to the doctor and attendants is proposed, wherein monitor of the RMD/Dig doctor software is used. The proposed monitor, Wi-Fi/LAN bi-directional integration is available. If the monitor detects any irregularities in the measuring parameters, it will display an abnormality indicator and an alarm.

By monitoring heart activity (ECG), blood pressure (BP), oxygen saturation (SpO2), and temperature, the patient monitor system collects health data (TEMP). It also measures pulse rate, blood pressure, and oxygen saturation of blood pressure all in one gadget. It delivers exceptional reliability in terms of avoiding data loss and ensuring data security, integrity, and provenance by incorporating the wireless connection (Wi-Fi) as part of the overall system design.

This Patient Monitor includes added capabilities such as video and audio calling. If your computer is connected to a local area network, you can also access the display of the monitor. This patient monitor can provide precise measurements of various health parameters such as SpO2, Pulse rate, Heart Rate, ECG, temperature, invasive blood pressure (IBP), and Respiration rate of patient.

There are numerous variants, modifications, additions, and enhancements that can be made to the embodiments described above. These variants, alterations, additions, and enhancements are thought to be within the scope of the invention.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A portable patient health monitoring system, the system comprising:
a plurality of sensor input lids engaged to the fingertips of a person/patient for detecting a set of health parameters;
a controlling unit for generating an alert signal upon comparing the detected set of health parameters to the threshold values stored in a database;
a communication device having a bi-directional integration facility for transferring the alert signal to a respective alarm indicator from a plurality of alarm indicators for indicating an imbalance of respective health parameters from the set of health parameters; and
a monitor for displaying the detected set of health parameters and indicating the imbalanced health parameters from the set of health parameters, wherein the graphical user interface allows checking the last pathology & Biochemistry, ABG report.

2. The system of claim 1, comprises a camera for supporting video calling and real-time data transfer of patients to the doctor, and attendants using an RMD/Dig doctor user interface.

3. The system of claim 1, wherein the set of health parameters is selected from a group of exact oxygen saturation (SpO₂), Pulse rate, Heart Rate, electrocardiography (ECG), temperature, Invasive Blood Pressure (IBP) and Respiration rate of the patient.

4. The system of claim 1, comprises a graphical user interface for displaying the detected set of health parameters and indicates the imbalanced health parameters from the set of health parameters, wherein the graphical user interface allows checking the last pathology & Biochemistry, ABG report remotely.

5. The system of claim 1, comprises an internet medium preferably a Wi-Fi to allow clinicians to make informed decisions, identify, and alert.

6. The system of claim 1, wherein the detected set of health parameters is converted from analog data into digital data using serial communication.

7. The system of claim 1, comprises an audio calling facility through the graphical user interface or through the communication module.

8. The system of claim 1, wherein said system is optionally paired with a user computing device to support real-time video calling with the doctor.

9. The system of claim 1, comprises a cloud server platform wirelessly connected to the controlling unit for storing the database of the threshold values.
